(19) United States
(12) Patent Application Publication
(10) Pub. No.: US 2015/0314134 A1
(43) Pub. Date: Nov. 5, 2015

(54) METHOD OF GENERATING LIFE SUSTAINING ENERGY IN THE TERMINAL AND RESPIRATORY BRONCHIOLES SYSTEM (TRBS)

(71) Applicant: Krzysztof Slawomir Nowicki, Staten Island, NY (US)

(72) Inventor: Krzysztof Slawomir Nowicki, Staten Island, NY (US)

(21) Appl. No.: 14/268,036

(22) Filed: May 2, 2014

(51) Int. Cl. A61N 5/06; A61H 23/00

(52) U.S. Cl. CPC A61N 5/0603; A61H 23/00; A61N 5/0613; A61N 2005/0604; A61N 2005/067

(57) ABSTRACT

The TRBS model of respiratory physiology is based on the water dissociation by low power radiation, emitted from CO₂ to hemoglobin as organic semiconductor. The unit is composed of three modules: (a) The Tunable Microcavity Module—in the terminal bronchioles, (b) The Compressor Module—in the respiratory bronchioles, (c) The Reservoir Module—in the alveolar sacs. Two types of optical processes lead to energy amplification: Spontaneous Emission and Thresholdless Emission in a Tunable Microcavity. The tunable microcavity is covered by a surfactant that serves as a multilayer reflective surface. The vagal nerve innervates the microcavity and the muscles contract (Q factor). The compressor controls CO₂ cooling. Collisions of colder CO₂ molecules with warmer homonuclear N₂ molecules, excited to a vibrational metastable level, which leads to the population inversion via vibrational excitation of the CO₂ that is necessary for microcavity lasing. The vibrational modes lattice transports energy to cells and genome.

### FIELD OF INVENTION

Respiratory Physiology, Anaesthesia, Infrared Spectroscopy, Optoelectronics, Optical Microcavities Description of the unrecognized process in the Terminal and Respiratory Bronchioles region in the lungs, as a life sustaining source of infrared energy, responsible for the non-diffusion hemoglobin saturation by endogenous oxygen and the body cells energization.

### ANATOMICAL AND PHYSIOLOGICAL BACKGROUND

### Introduction

The modern understanding of respiratory physiology is inconsistent. The mechanism of passive oxygen diffusion in the lungs is unclear regarding the short physiological transition time of oxygen (from 0.8 sec—at rest to 0.25 sec—with effort) [1] and the complicated structure of the lower airways. The non-diffusion model of hemoglobin saturation by oxygen is difficult to determine because the emission of heat from the human body is mostly in the infrared range; however, the skin becomes a barrier for a bandwidth between 9-11 μm of the mid-infrared range. The high number of last generation of terminal bronchioles units (˜1 billion); the small size and short distance between the alveolar sacs, respiratory and terminal bronchioles, arterioles and pulmonary capillaries appear challenging for physiological research. The terminal bronchioles are the last generations (the 14^{th}) which conduct inhaled ambient air. Inhaled oxygen appears in the terminal bronchioles but there is not enough proof to conclude that it is present within the intact respiratory bronchioles and alveolar sacs, because the oxygen's rapid uptake (physiological increase oxyhemoglobin), appears even within the pulmonary arterioles that are located before the pulmonary capillaries. [1] Confining the haemoglobin within the RBCs reduces the oxygen diffusing capacity by 40% in comparison with free haemoglobin in solution. [1] Practically, the cell's regular metabolism (metabolic heat production) is masking any subtle output of power in the order of the μW magnitude so non-diffusion hemoglobin saturation by O₂ appears as an oxygen passive diffusion process.

Turbulent Air Flow

The most efficient way that some fresh gas will reach the end of a tube is by laminar flow. The process gives the best alveolar ventilation in the shortest amount of time because during gas flow, an advancing cone forms a parabolic shape. The turbulent flow is characteristic for irregular or branched tubes. An irregular movement is superimposed on the general progression along the tube, with a square front replacing the cone front of laminar flow. The square front means that no fresh gas can reach the end of a tube until the amount of gas entering the tube is almost equal to the volume of the tube. [1] The qualification of the nature for gas flow is describing by the Reynolds' number. Turbulent flow is more effective than laminar flow in purging the contents of a tube. Frictional forces between the tube wall and gas become more important in turbulent flow. In human lungs, the turbulent flow appears in all, 14 generation of bronchi and bronchioles (air conduction) which have a dichotomy system (first 6 generations and 9^{th}-14^{th} generations) or trifurcation branches (7^{th}-8^{th} generations). The anatomical structure of airways supports a pattern of multiplying the airways. The diameter of the adult airways decreases from the trachea size of 18 mm, through the main bronchi that is 12 mm, the lobar bronchi 8-5 mm, segmental bronchi 4 mm, small bronchi 3-1 mm, Terminal bronchioles 1-0.7 mm, Respiratory bronchioles 0.4 mm, Alveolar ducts measure 0.3 mm, to a diameter of 0.2 mm in the alveoli. In succeeding generations, the number of bronchioles increases far more rapidly than the caliber diminishes. Therefore the total cross-sectional area increases until, in the terminal bronchioles, it is about 100 times the area at the level of the large bronchi. Moreover, the number of air passages increase from a single trachea to 4,000,000 alveolar ducts in one pulmonary acinus. [1] Turbulent airflow supports CO₂ purging from the collateral ventilation outlets in the bronchioles and leads to an increase effectiveness of air molecules collisions. Importantly, development of children's lungs is based on multiplying the terminal bronchioles, respiratory bronchioles and alveolar sacs until the child is seven years old. After this age, the growth of the lungs is based on an enlargement of all these parts.

Residual Volume (RV)

Residual volume (RV) is the volume remaining after a maximal expiration. In the young, RV is governed by the balance between the maximal force generated by the expiratory muscles and the elastic forces opposing the reduction of lung volume. However, in older subjects, closure of the small airways may prevent further expiration. [1] By definition, RV is the O₂ reservoir, but also clocks fresh air delivery directly to the alveolar sacs during inhalation. This means that the alveolar CO₂ purging is an unmarked delay, especially in older subjects. The rate of oxygen transfer is not proportional to the alveolar/capillary pO₂ gradient at any point along the pulmonary capillaries. [1] In healthy young adults breathing air, the alveolar/arterial pO₂ differences does not exceed 2 kPa (15 mmHg) but rises to above 5 kPa (37.5 mmHg) in aged but healthy subjects. The consequences of unclear gases distribution in the lungs appears as a side effect during anesthesia. The major differences are between the awake and the anaesthetised states. [1] Paralysis and artificial ventilation do not greatly alter the parameters of gas exchange, despite the different spatial distribution of ventilation. Uniformity of ventilation distribution and perfusion is decreased by anaesthesia. [1] The magnitude of the change is age-related and may be affected by the inspired oxygen concentration and the anaesthetic agents used. The increase in alveolar dead space appears to be due to an increased distribution of ventilation to areas of high, but not continuing infinite ventilation and perfusion ratios. The problem of atelectasis and an associated venous admixture increased to a mean value of about 10% that appears related to age and is minimal in the young. The increased venous admixture during anaesthesia is partly due to an increase in a true intrapulmonary shunt because of the appearance of atelectasis and partly due to an increased distribution of perfusion to areas of low ventilation and perfusion ratios. [1] General anesthesia associated with increased risk of Alzheimer's disease, because anesthesia induces hypothermia, which leads to overt tau hyperphosphorylation in the brain of mice regardless of the anesthetic used. [32][33][34] The hyperbaric oxygen therapy used as an intuitive consequence created to improve the passive diffusion, gives side effects, which are often mild and reversible, if the pressures do not exceed 300 kPa and the length of the treatment is less than 120 minutes. Under such conditions, hyperbaric oxygen therapy is safe, but can be severe and life threatening if applied for a longer period of time. [1]

Collateral Ventilation

Collateral ventilation is defined as the ventilation of alveolar structures through passages or channels that bypass the normal airways. Four collateral pathways have been described: the interalveolar, the bronchioloalveolar, the interbronchiolar and the interlobar communications. Collateral channels can provide alternative pathways in various human pathologies. The phenomenon of collateral ventilation explains several clinical observations in human lungs such as the absence of atelectasis following complete bronchial obstruction. [1] The pattern of the responses to local hypercapnia and hypocapnia, along collateral channels and along the airways are functionally and structurally similar. [2] The passive diffusion model shows collateral ventilation as alternative pathways without any important physiological functions; however, maintains patency in various human pathologies.

The Significant Lack of Cartilage in the Terminal Bronchioles Wall

An important change occurs at about the 11th generation where the internal diameter measures about 1 mm. Cartilage disappears from the wall below this level and ceases to be a factor in maintaining patency. [1] The resistance of the bronchioles can increase to very high values when their strong helical muscular bands are contracted. In normal lungs, respiratory resistance is controlled by changes in airway diameter, mainly in the small airways and bronchioles; therefore the caliber of the airways below 11th generation is influenced mainly by lung volume. [1] The number of bronchioles increases far more rapidly than the caliber diminishes. The total cross-sectional area increases until, in the terminal bronchioles about 100 times the area at the level of the large bronchi. From the point of view of the passive diffusion model, these structural changes are the risk-factor of an airway higher resistance leading to a fall in the patency.

The Epithelial Cells in the Bronchioles

In the bronchi, the cell height begins to reduce and tends toward developing into cuboidal shaped epithelial cells, before gradually flattening further throughout the pulmonary acinus and merging with the alveolar epithelial cells. [1] This tendency to flatten cells can better support more flexibility of the walls than in maintaining patency. The ciliated epithelial cells almost disappears close border with the respiratory bronchioles. [1] The airways epithelium is itself capable of directly generating a reactive oxygen and reactive nitrogen species (ROS/RNS). [3][4] The precise biologic mechanisms responsible for these associations have yet to be fully established. The antioxidant activity of surfactant “in vivo” remains unclear.

Helical Bands of Smooth Muscle in Terminal Bronchioles and Corresponding Arteries

The role of the strong helical band of smooth muscle along the terminal bronchioles is unclear. [1] To be only a factor in maintaining patency is not an adequate explanation for the structure's function in this area. The passive diffusion model does not explain enough of the physiological circumstances that supports the contraction or relaxation of the smooth muscles strong helical bands and their frequency in regard to ventilation. Some post-mortem images of the lungs, especially of the terminal bronchioles region, show a contraction of the smooth muscles helical bands, which are formed in a very narrow space, equivalent to a microcavity. This process has been called a post-mortem artifact because it does not support the passive diffusion pattern. [1] The role helical bands of smooth muscles in the corresponding arteries can be better explained in regard to blood perfusion. The media of the pulmonary arteries is about half as thick as in systemic arteries of corresponding size and carries only one-sixth of the systemic arterial pressure. In the larger vessels, it consists mainly of elastic tissue but in the smaller vessels it is mainly muscular, the transition exists in vessels of about 1 mm, in diameter. The pulmonary arterioles occur in an internal diameter of 0.1 mm and lie near the terminal bronchioles. These vessels differ radically from their counterparts in systemic circulation, because they are virtually devoid of muscular tissue. There is a thin media of elastic tissue separated from the blood by endothelium. [1] The scheme of structure and distribution of pulmonary arteries (which lie close to the corresponding air passages in connective tissue sheaths), support a regulatory mechanism of perfusion sped by vascular resistance. The faster or slower blood perfusion is occurs leads to an increase or decrease of hemoglobin (Hb), contact with O₂ and effectiveness of CO₂ purging.

The Rings of Smooth Muscles in Alveolar Ducts and Respiratory Bronchioles

The explanation of function regarding structure of a well-defined smooth muscles layer, with bands that loop over the opening of the alveolar ducts and the mouths of the mural alveoli in the respiratory bronchioles is complicated. [1] Patency maintenance is not fully understood. The main question is why do the bands of smooth muscles appear in this location where the fast air flow and diffusion are most important during the inhalation and exhalation? What kind of frequency, with regards to contraction-relaxation appears during the inspiration and expiration? The precise biologic mechanisms responsible for these associations have yet to be fully established.

Surfactant's Fatty Acids Layer

The low-surface tension of the bronchiolar and alveolar lining fluid and its dependence on alveolar radius are due to the presence of the surface-active material known as the surfactant. [1] 90% of the surfactant is built from lipids. The remainder consists of proteins and a small amount of carbohydrates. Most of the lipids are phospholipids, of which some 70-80% is dipalmitoyl phosphatidyl choline, which is responsible for the effect of surface tension. The surfactant phospholipid is known to exist “in vivo” in both monolayer and multilayer forms. The phospholipid alternates between these two forms during the respiratory cycle. This aspect of surfactant function is dependent on the presence of surfactant proteins B (SP-B) a small hydrophobic protein, which can be incorporated into a phospholipid monolayer and surfactant proteins C (SP-C) a larger protein with a hydrophobic central portion allowing it to span lipid bilayers. Some phospholipids have significant activity in the bandwidth 9-11 μm. The most important problem is that fatty acids are hydrophobic and straight, lying parallel to each other and projecting into the gas phase. The other end of the molecule is hydrophilic and lies within the alveolar and bronchiolar lining fluid. The consequences of this structure and the location are not compatible to the gas law requirement for the passive diffusion process. Henry's law describes the solution of gases in liquids with which they do not react. [1] The number of gas molecules dissolving in the solvent is directly proportional to the partial pressure of the gas at the surface of the liquid, and the constant of proportionality is an expression of the solubility of the gas in the liquid. This is constant for a particular gas and a particular liquid at a specific temperature but usually falls with rising temperatures. This hydrophobic layer projects into the gas phase and blocks liquid transudation with solved gas molecules through the surfactant layer. Moreover, the hydrophilic end of the molecules of surfactant structure better support CO₂ diffusion from the pulmonary capillary to the alveolar and bronchiolar space. Taking into consideration that a very low coefficient of solubility of O₂ into the water and plasma, when compare to the CO₂ solubility; the lack of any known carriers supporting O₂ diffusion, the situation becomes unclear, regarding the physiological transit time to O₂ (i.e., in the range of 0.8-0.25 sec.)

Distribution of Gases in Alveolus Space

The diameter of the average human alveolus is in the order of 200 μm. Fresh oxygenated air, is attained at the center of the alveolus, but at the periphery of the space, which is especially close to the capillary, oxygen diffusion is highest, and there is reduced O₂ tension. The delay of the distribution time for oxygen molecules consist in a longer path between the middle points and the peripheral points of the alveolus space where the gradient of partial pressure is responsible for diffusion effectiveness. The physical pattern of the gas distribution layers, delays the oxygen diffusion because the tension gradient between the central and peripheral space of alveolus is differently related to the calculated oxygen transition time. However, respiratory physiology assumed that the overall efficiency of gas exchange within the lungs suggests that mixing must be complete within less than 10 milliseconds. [1] In practice, this startling fact is disregarded and the alveolar gas is considered of a normal composition as it is uniformly mixed. A precise calculation is impossible because of the complex alveolus geometry. The alveolar air equation implies that a hyperbolic relationship exist between the alveolar pO₂ and the alveolar ventilation. [1] As ventilation is increased to the normal level, the alveolar pO₂ rises asymptomatically towards the pO₂ of the inspired gas but it never reaches the same level. Comparatively, the changes in ventilation above the normal levels have little effect on alveolar pO₂. In contrast, the changes in ventilation below the normal level may have a very marked effect. At very low levels of ventilation, the alveolar ventilation becomes critical and small changes may precipitate severe hypoxia. [1]

The Length of the Diffusion Path, Diffusing Capacity and Transit Time of Oxygen

The physiological O₂ diffusion transit time in humans is very short range of 0.8-0.25 sec., [1] and is difficult to explain from a physics point of view. For this reason scientists searched for any carrier that supported the acceleration of O₂ transport through the alveolar and capillary wall. Cytochrome P-450 has been examined but it is a not well supported process for explanation. The situation is actually still more complicated as quick-frozen sections of lung show that the color of hemoglobin begins to alter to the color red of oxyhemoglobin within the pulmonary arterioles before the blood has even entered the pulmonary capillaries. Furthermore, pulmonary capillaries do not cross a single alveolus but may pass over three or more. [1] The actual path between alveolar gas (exempt surfactant) and pulmonary capillary blood of the active part of the tissue barrier is about 0.5 μm, containing two pairs of lipid bilayers (hydrophobic) separated by the interstitial space. Human pulmonary capillaries are 14 times the thickness of the tissue barrier, it is clear that the diffusion path within the capillary is likely to be much longer than the path through the alveolar/capillary membrane. A complex pattern of diffusion gradients is established within the plasma that depends on the oxygen tension in the alveolus and the number of the red blood cells (RBCs) present. Confining the haemoglobin within the RBCs reduces the oxygen diffusing capacity by 40% in comparison with free haemoglobin in solution. [1] The result of many experiments suggested that diffusion is the main process limiting O₂ uptake and release by RBC, the finite reaction kinetics of O₂ with hemoglobin exerting a smaller limiting effect. However, there is now good evidence that the rapid uptake of O₂ by the RBCs causes depletion of gas in the plasma layer immediately surrounding the RBC. This is referred to as the “unstirred layer” (USL). This phenomenon is most likely to occur at a low packed cell volume (PCV) when adjacent RBCs in the pulmonary capillary have more plasma between them. [1] Unfortunately the stopped-flow technique bases on 37° C. temperature generates infrared wavelength (bandwidth contains fraction of ˜9.34 μm of wavelength) and can excite hemoglobin regards to photosensitivity and organic semiconductor properties hence the “unstirred layer” conditions can not explain the passive diffusion presence. Additionally, the phenomenon creates a problem of very low solubility coefficient of oxygen in plasma with the diffusion path through plasma in the capillary and RBCs membranes and liquids. Significantly, the transit time (0.8-0.25 sec) when compared to the very low solubility coefficient of oxygen (0.003 ml/100 ml, pO₂=1 mmHg in temp 37° C.) [1] appears as an unclear passive diffusion support factor. According to Henry's law, the coefficient of solubility of O₂ in plasma is 0.3 ml/100 ml in temp. 37° C., pO₂=100 mmHg., with a mean of 3.0 ml O₂ in 1000 ml of blood (0.3%/1 liter) can physically dissolved in the diffusion path near pO₂=100 mmHg. Beside the calculation based on Henry's law, the real measured difference of O₂ volume between arterial and venous blood is ˜60 ml (200-140 ml O₂/1000 ml of blood). The conclusion is that O₂ plasma's diffusion capacity has a 95% deficiency, this means that 57 ml O₂/1000 ml is below the needs, because of the very low the coefficient of solubility (0.3 ml/100 ml in temp. 37° C., pO₂=100 mmHg). Moreover, the bi-directional movement of O₂ and CO₂ between the RBC and pulmonary capillary wall can significantly decrease the tension of O₂. It is known that the solubility coefficient of CO₂ in plasma can exceed 24 times the coefficient of solubility of O₂ and diffusing capacity of CO₂ is 20.5 times greater than oxygen. [1] The role of KCl, NaCl, albumins, globulins and glucose in plasma does not support the model of passive diffusion process.

Peripheral Chemoreceptors Monitoring O₂ Tension in Blood Vs. Medullary Chemoreceptors Monitoring CO₂ Tension in Blood

It is wide accepted that the medulla is the area of the brain where the respiratory pattern is generated and where the various voluntary and involuntary demands on respiratory activity are coordinated. [1] Respiratory neurons in the medulla are mainly concentrated in two anatomical areas, the ventral and dorsal respiratory groups, which have numerous interconnections. Early in pregnancy, the fetal brainstem develops a respiratory center, which produces uninterrupted rhythmic breathing activity. The medullary chemoreceptors monitor a rise in arterial P_{CO2}, respiratory depth and rate increase until a steady state of hyperventilation is achieved. About 85% of the total respiratory response to inhaled CO₂ originates in these medullary chemoreceptors. [1] This means that CO₂ plays a majority role as a stimulus of respiratory activity. However, the central chemoreceptors are separate from the respiratory neurons of the medulla although they are located only a short distance away. Other areas of the CNS that display increased neuronal activity with CO₂ stimulation include the midline pons, small areas in the cerebellum and the limbic system, which remains unclear. On the other hand the peripheral chemoreceptors located close to the bifurcation of the common carotid artery appeared sensitive to a fall in pO₂ levels, and a rise in pCO₂, H⁺ concentration, and tend to fall in the perfusion rate. [1]

Innervation of Airway Tree in Animals and Humans

The distribution of nerve fibers and neuroendocrine cells, within the intrapulmonary airways is highly heterogeneous, varying between the airway levels and locally, within a specific airway level. The role of these nerves in the development of airway hyper-reactivity and inflammation cannot be assessed before there is an understanding of the distribution of these fibers. The anatomical associations between nerve fibers and other airway structures should be explored in detail before speculations are made that functionally link nerves to other airway cell types, such as neuroendocrine cells. [14] [15]. The numbers of myelinated fibers in the airway diminish as they pass scattered ganglion cells, along the bronchial system. In the rhesus monkey lungs [14] the extrachondral and subchondral plexuses of nerves were found to be interconnected and to contribute to the perimuscular varicose nerve plexus of the bronchi and bronchioles. These nerve plexuses were found to extend as far as the respiratory bronchioles. In the bronchial submucosa, there are Acetylcholinesterase-positive (AChE-positive) nerve plexuses which arise from three sources: (A) the adventitial plexus in bronchioles, or the subchondral plexus in bronchi, (B) the perimuscular nerve plexus, and (C) AChE-containing nerves, associated with the bronchial artery. The submucosal plexus appears to innervate the acinar submucosal glands in the bronchi, as well as continuing as central nerves in the mucosal folds. In the bronchioles, the nerves in the mucosal fold are close to the mucosa. The parasympathetic efferent nerves are supplied to the airways via the vagus nerve. [15] There are a large number of different types of receptors in the lungs that are sensitive to inflation, deflation, and mechanical and chemical stimulation and afferents, which are mostly conducted by the vagus nerve, although some fibres may be carried in the sympathetic nerves. Vagal preganglionic fibers enter the lungs and terminate in the ganglia located within the airways themselves. Studies with acetylcholinesterase stains indicate a dense cholinergic innervation of bronchi in all species studied. The airways of dogs, cats, rabbits and healthy humans are tonically constricted and this tone is maintained by vagal efferent nervous activity. When the vagus nerves are stimulated, constriction occurs from the trachea to the large bronchioles, with maximum constriction occurs in the bronchi of intermediate size. Significantly, the terminals bronchioles and alveolar ducts are unaffected by reflex bronchoconstriction, so lung compliance is usually unchanged unless bronchoconstriction is severe. The Vagal nerve has connectivity with the central areas and its stimulation is activated in the anterior thalamus (part of limbic system), posterior temporal cortex, putamen and inferior cerebellum. [22] When severed, the vagal nerve has fast regeneration of nerve fibers and recurrence of conduction. [1] Lung transplantation inevitable disrupts innervation, lymphatic and the bronchial circulation and present no evidence that reinnervation occurs in human patients [1]; however in the canine model, vagal stimulation has been observed to cause bronchoconstriction 3-6 months after lung reimplantation, and sympathetic reinnervation has been demonstrated after 45 months. Most patients report mild to moderate hypercarbia for the first several weeks after transplantation that later gradually normalizes. This is either because of inadequate feedback signals from the respiratory muscles, due to the reduced respiratory effort required after transplantation or because there is delay in resetting the central chemoreceptors, patients frequently experience a sense of hypoxia in the postoperative period and will often respond by hyperventilation. Repeated blood gas determinations during these episodes are frequently in the normal range and have not shown hypoxemia. [17] The perioperative and postoperative mortality after lungs transplantation is significant. The condition of the recipient is further compromised by the administration of immunosuppressive therapy. After a period of five years, lung transplantation survival rate is less than 50% [1] and after ten years the survival rates dramatically further decline. Acute rejection appeared as a problem associated with increased mortality, but chronic rejection in the lung manifests itself as an obliterative bronchiolitis syndrome, the origin of which is not clear but which occurs in up half of all patients, normally reported more than a year after transplantation.

CO₂ Vs. O₂ Stored Capacity in Human Body

The amount of stored CO₂ in humans is very high. Most of CO₂ is stored in bones as carbonates or dissolved in fats. In the human body most liquids especially blood contain about 500 ml/1 liter of CO₂. Distribution of carbon dioxide within the blood is mostly as bicarbonate ions. However, the bicarbonate ions are formed by ionization of carbonic acid which is under physiological conditions far to the left of the equilibrium of given reaction [CO₂+H₂O [symbol] H₂CO₃] because it is slow non-ionic reaction and requiring a period of minutes for equilibrium to be attained. Carbonic anhydrase (CA) catalyses this reaction in both direction but is not CA activity in plasma where bicarbonate are the main fraction (24.4 mmol·l⁻¹ or 544 ml/1 liter of blood). [1] The problem remains unclear because most of carbon dioxide enters and leaves the blood as CO₂ itself. [1] Published work shows some disagreement on the value of the equilibrium constant, but it seems likely that less than 1% of the molecules of carbon dioxide are in the form of carbonic acid. There is a very misleading medical convention by which both forms of carbon dioxide in equation above are sometimes shown as carbonic acid. [1] In contrast, all liquids in the human organism stores less than 2 ml/1 liter of O₂, an exemption is the arterial blood that contains 200 ml/1 liter of O₂ combined with Hb. [1] However, Hb is the most efficient chemical carrier, with more than 0.5 kg required to carry 1 g of oxygen. The concentration of Hb in the blood far exceeds the concentration of any protein in any other body fluid. Even so, the quantity of oxygen in the blood required for sustaining life is barely sufficient for three minutes of metabolism in a resting state. It is a fact of great clinical importance that the body oxygen stores are so small.

Organic Semiconductors, Oxyhemoglobin and Dissociation Curve

An organic semiconductor is an organic material with semiconductor properties. [19] The conductivity of a semiconductor is generally intermediate, but varies widely under different conditions, such as exposure of the material to electric fields or specific frequencies of light, and, most importantly, with temperature and consideration of the semiconductor compositional material. In semiconductors, electrical conductivity increases with increasing temperature. Semiconductivity may be exhibited by single molecules, short chain (oligomers) and organic polymers. Semiconducting small molecules (aromatic hydrocarbons) include the polycyclic aromatic compounds pentacene, anthracene, and rubrene. Polymeric organic semiconductors include poly(3-hexylthiophene), poly(p-phenylene vinylene), as well as polyacetylene and its derivatives. Polypyrroles are conducting polymers of the rigid-rod polymer host family, all basically derivatives of polyacetylene. Polypyrrole was the first polyacetylene-derivative to show high conductivity. Polypyrrole (PPy) is a chemical compound formed from a number of connected pyrrole ring structures. For example, a tetrapyrrole is a compound with four pyrrole rings connected; methine-bridged cyclic tetrapyrroles called porphyrins. Porphyrins are a group of organic compounds of which many occur in nature. One of the best-known porphyrins is heme, the pigment in red blood cells; heme is a cofactor of the protein hemoglobin. Porphyrins are heterocyclic macrocycles composed of four modified pyrrole subunits interconnected at their a carbon atoms via methine bridges (═CH—). Porphyrins are aromatic. Hemoglobin and myoglobin are two O₂-binding proteins that contain iron porphyrins. The hemoglobin molecule consists of four amino acid chains, each carrying a heme group. The four chains of the haemoglobin molecule lie in a ball, like a crumpled necklace. However, the form is not random and the actual shape (the quaternary structure) is of critical importance and governs the reaction with oxygen. [1] The shape is maintained by loose electrostatic bonds between specific amino acids on different chains and also between some amino acids on the same chain. The heme group is also attached by an electrostatic bond to the histidine residue in position 58 and also by non-polar bonds to many other amino acids. This forms a loop and places the heme group in a crevice, the shape of which controls the ease of access for oxygen molecules. The electrostatic forces are important in maintaining a subtle change with age in the conformation of the hemoglobin molecule. The organic semiconductor characterizes by a charge transport that is dependent on the π-bonding orbital and quantum mechanical wave-function overlap. Quantum tunneling allows the electron simply to go through the potential barrier instead of going all the way over it because of the electron wave nature. Because of the quantum mechanical tunneling nature of the charge transport, and its subsequent dependence on a probability function, this transport process is commonly referred to as hopping transport. [20] Hopping of charge carriers from molecule to molecule depends upon the energy gap between highest occupied molecular orbital (HOMO) and lowest unoccupied molecular orbital levels (LUMO). The temperature and electromagnetic field (quantum coherent energy radiation) affects this phenomenon across the system. The mechanism of HOMO-LUMO gap and quantum mechanical tunneling appears in organic semiconductors like hemoglobin. In a natural environment a tetrapyrroles (bilins) appearance in Cryptophytes which are eukaryotic algae that live in marine and freshwater environments. [27][28] The Cryptophyte photosynthetic antenna proteins (phycobiliproteins) exhibit exceptional spectral variation between species because they use mainly tunable linear tetrapyrroles (bilins) for light-harvesting. The evolutionarily related light harvesting proteins isolated from marine cryptophyte algae, which reveal an exceptionally long-lasting excitation oscillations that have distinct correlations and anti-correlations, even at ambient temperature [26][27][28] These observations provide compelling evidence for quantum coherent sharing of electronic excitation across the 5-nm-wide proteins under biologically relevant conditions, suggesting that distant molecules within the photosynthetic proteins are ‘wired’ together by quantum coherence for more efficient light-harvesting in cryptophyte marine algae. Another remarkable feature of Cryptophytes is that they can photosynthesize in low-light conditions, which suggest that, the absorption of incident sunlight by phycobiliprotein antennae in the intrathylakoid space and the subsequent transfer of that energy among these proteins and eventually to the membrane-bound photosystems is particularly effective. According to Sholes, [27] there is direct evidence that remarkably long-lived wavelike electronic quantum coherence plays an important part in energy transfer processes during photosynthesis, which can explain the extreme efficiency of the energy transfer because it enables the system to sample all the potential energy pathways, with low loss, and choose the most efficient one. Photolysis is part of the photons-dependent reactions of photosynthesis. The electronic properties of organic semiconductors such as ionization energy (E_{I}) of molecule means the energy needed to remove electrons from atom. The electron-deficient reaction center of photosystem II (P680*) is the strongest biological oxidizing agent yet discovered, which allows it to break apart molecules as stable as water. [29] The general reaction of photosynthetic photolysis can be given as: [H2X+2 photons (light)→2e⁻+2H⁺+X]. The chemical nature of “X” depends on the type of organism. In purple sulfur bacteria, hydrogen sulfide (H₂S) is oxidized to sulfur (S). In oxygenic photosynthesis, water (H₂O) serves as a substrate for photolysis resulting in the generation of free oxygen (O₂). Photolysis of water occurs in the chloroplasts of green algae, plants and the thylakoids of cyanobacteria. [26] [27] [28]

Two ways leading to Hb saturation by oxygen: direct contact with oxygen (real diffusion) or rapid changes of the electromagnetic field around the RBC, (with confined Hb) e.g., exposure to coherent waves. Hb is sensitive to stimulation by electromagnetic field of a wide range of coherent wavelength: 337 nm (N₂ laser) [6], 632.7 nm (He—Ne laser) [8], 9.676 μm (wavelength tunable CO₂ laser). [7] In the case of mid-infrared, single photons usually are not energetic enough for direct photodissociation of molecules. However, after absorption of multiple infrared photons, a molecule may gain internal energy to overcome its barrier for dissociation. In that case, such a mid-infrared multiple photon dissociation can be achieved by applying long interaction times the molecule with a radiation field that does not have the possibility of rapid cooling. The responds to quantum coherence stimulus supports a pattern of the oxygenic photosynthesis. The oxyhemoglobin dissociation curve (The Bohr Effect) [1] describes the relation between the oxygen saturation or content of hemoglobin and the oxygen tension at equilibrium. The dissociation curve is not sensitive to inhalation and exhalation oscillations because of the residual volume (RV). However, very low oscillation of pCO₂ and pH of arterial blood, when observed in the same phase are consistent with breathing and is increased during effort. [1] The oxyhemoglobin dissociation curve is shifted to the right when the temperature rises high, pH is lower and pCO₂ is increased. The shift to the right is beneficial for tissue oxygenation. It is a general rule that a shift to the right (increased P₅₀) will benefit venous pO₂, provided that the arterial pO₂ is not critically reduced. The shift to the left corresponds to a lower then normal temperature, higher pH, and decrease of pCO₂. The role of 2,3-diphosphoglycerate (2,3 DPG), responsible for oxygen affinity to Hb and the impact on displacing the dissociation curve to the right was observed. However, the likely effects of changes in P₅₀ mediated by 2,3 DPG seem to be of marginal significance in comparison with changes in arterial pO₂, acid-base balance and tissue perfusion. [1]

The Marshall-Whyche's Equation

There is no evidence that arterial pCO₂ is influenced by age in the healthy subject. In contrast to the arterial P_{CO2}, the arterial P_{O2} shows a progressive decrease with age.

Arterial P_{O2}=13.6−0.044×age in years (kPa) or

Arterial P_{O2}=102−0.33×age in years (mmHg)

Concerning a regression line, there is 95% confidence limits of +/−1.33 kPa (10 mmHg) with only 5% of normal patients who will lie outside these limits. [1]

Rejuvenation of Aged Progenitor Cells by Exposure to a Young Systemic Environment

Researchers [16] examined the efficacy of muscle regeneration in young 2-3 month old transgenic mice and aged 19-26 month old transgenic mice in heterochronic and isochronic pairings (C57Bl/Ka-Ly5.2, b-actin-eGFP, and C57Bl/6 mice). The study reported that there are systemic factors supporting robust regeneration of tissues in young animals and/or inhibit regeneration in older animals, and that these factors act to modulate the key molecular pathways that control the regenerative properties of progenitor cells. To examine the influence of systemic factors on aged progenitor cells from examined tissues, parabiotic pairings were established between young and old mice (heterochronic parabioses), exposing old mice to factors present in young serum by a shared circulatory system. The controls were established with parabiotic parings between two young mice or two old mice (isochronic parabioses). The results suggest that the age-related decline of progenitor cell activity can be modulated by systemic factors that change with age. The core temperature was not measured.

Sleep

Sleep is a naturally recurring state characterized by a reduced or absent consciousness, relatively suspended sensory activity, and an inactivity of nearly all voluntary muscles. In mammals and birds, sleep is divided into two broad types: rapid eye movement (REM) and non-rapid eye movement (NREM or non-REM) sleep. The American Academy of Sleep Medicine (AASM) further divides NREM into three stages: N1, N2, and N3, the last of which is also called delta sleep or slow-wave sleep (SWS). [25] The time frame of sleep is different for the newborn i.e., up to 18 hour and for adults a 7-8 hour. Sleep is also a heightened anabolic state, accentuating the growth and rejuvenation of the immune, nervous, skeletal and muscular systems. It is observed in all mammals, all birds, and many reptiles, amphibians, and fish. The purposes and mechanisms of sleep are only partially understood and are the subject of intense research. Sleep is often thought to help conserve energy, but actually decreases metabolism only about 5-10%. Sleep disturbances also affect temperatures. Normally, body temperature drops significantly at a person's normal bedtime and throughout the night. Short-term sleep deprivation produces a higher temperature at night than normal, but long-term sleep deprivation appears to reduce temperatures. Insomnia and poor sleep quality are associated with smaller and later drops in body temperature. [35][36] Wound healing has been shown to be affected by sleep. A study conducted by Gumustekin et al., [23] in 2004 shows that sleep deprivation hindering the healing of burns in rats. It has been shown that sleep deprivation affects the immune system. In a study by Zager et al., reported in 2007, [24] rats were deprived of sleep for 24 hours. When compared with a control group, the sleep-deprived rats' blood tests indicated a 20% decrease in the white blood cell count, reflecting a significant change in the immune system. It has been suggested that mammalian species, which invest in longer sleep times, are investing in the immune system, as species with the longer sleep times have higher white blood cell counts.

The Laser Physics Background: The Black Body Theory

In physics, a black body is defined as an idealized object that absorbs all electromagnetic radiation falling on it. [18] Blackbodies absorb and re-emit radiation in a characteristic, continuous spectrum. However, a black body emits a temperature-dependent spectrum of light. This thermal radiation from a black body is termed black-body radiation. In the blackbody spectrum, the shorter the wavelength, the higher the frequency, and the higher frequencies are related to the higher temperature. Thus, the color of a hotter object is closer to the blue-end of the spectrum and the color of a cooler object is closer to the red-end. At room temperature, black bodies emit mostly infrared wavelengths. The International Commission on Illumination (CIE), recommends the division of infrared radiation into the following three bands:

IR-A: 700 nm-1400 nm (0.7 μm-1.4 μm)

IR-B: 1400 nm-3000 nm (1.4 μm-3 μm)

IR-C: 3000 nm-1 mm (3 μm-1000 μm)

In the laboratory, black body radiation is approximated by the radiation from a small hole entrance to a large cavity, that has reached and is maintained at equilibrium. (This technique leads to the alternative term cavity radiation.) Any light entering the hole would have to reflect off the walls of the cavity indefinitely or to be absorbed. This occurs regardless of the wavelength of the radiation entering (as long as it is small compared to the hole size). The hole, then, is a close approximation of a theoretical black body and, if the cavity is heated, the spectrum of the hole's radiation (i.e., the amount of light emitted from the hole at each wavelength) will be continuous, strictly providing that the cavity must contain some near perfect black material body and that equilibrium has been reached and is maintained, but with these provisoes, it does not further depend on the other material in the cavity (as compared with the emission spectrum). The emissivity of a material (usually written & or e), is the relative ability of a material's surface to emit energy by radiation. It is the ratio of energy radiated by a particular material to the energy radiated by a black body at the same temperature. It is a measure of a material's ability to radiate absorbed energy and means its thermal emission deviates from the ideal of black body. A true black body would have a ∈=1 while any real object would have ∈<1. Emissivity is a dimensionless quantity, so it does not have units. Black body laws can be applied to human beings. For example, some of a person's energy is radiated away from the body in the form of electromagnetic radiation, most of which is infrared. The human skin emissivity is ∈=0.9, (close to the black body ∈=1). The Wien's Displacement Law can be applied to calculate the wavelength for skin temperature 36.6° C. Wavelength is equal to 9.36 μm. For lung temperature 38° C., the wavelength is 9.32 μm (IR-C band). It can be compared to a carbon dioxide bandwidth. When the body temperature is rising, the wavelength becomes shorter but, temperature in the range 41-42° C. (9.23-9.20 μm) usually is borderline for the well function of human proteins. However, it is equal to the shorter wavelength of the carbon dioxide bandwidth. With regard to human skin emissivity (∈=0.9), the Stefan-Boltzmann law can be applied to evaluate the power density of some regions the human body.

Infrared (IR) and Raman Spectroscopy

Infrared spectroscopy incorporates the fact that molecules absorb specific frequencies that are characteristic of their structure. These absorptions are of resonant frequencies, i.e., the frequency of the absorbed radiation matches the frequency of the bond or group that vibrates. The energy difference for transitions between the ground state (υᵢ=0) and the first excited state (υᵢ=1) of most vibrational modes corresponds to the energy of radiation in the mid-infrared spectrum 25 μm-2.5 μm (400-4000 cm⁻¹). [13] The energies are determined by the shape of the molecular potential energy surfaces, the masses of the atoms, and the associated vibronic coupling. The infrared spectrum of a sample is recorded by passing a beam of infrared light through the sample. Examination of the transmitted light reveals how much energy is absorbed at each wavelength. This can be done with a monochromatic beam, which changes in wavelength over time, or by using a Fourier transform instrument to measure all wavelengths at once. The Fourier transform is the operation that decomposes a signal into its constituent frequencies. From this, a transmittance or absorbance spectrum can be produced, showing at which IR wavelengths the sample absorbs. Analysis of these absorption characteristics reveals details about the molecular structure of the sample. When the frequency of the IR is the same as the vibrational frequency of a bond, absorption occurs. The changes of proteins structure are examined at Raman and IR spectroscopy depends on temperature, the solution concentration, pH, the ions intensity, etc. The absorption of many bonds or group vibrating in human body (the resonance frequency depends on temperature) is in range of 9-10 μm. For example it is compatible with: erythrocytes membrane phospholipids C—C groups for wavelength 9.2 μm, 9.4 μm, the helix proteins of muscles which vibrates in wavelength 9.6 μm, the neuronal fibers bands with vibration frequency C—H groups for wavelength 9.2 μm, and the plasma components i.e. glucose at spectral region 8.5-10 μm and wavelength 9.68 μm is characteristic for C—O—H bonds and 9.3 μm for C—H bonds, CO₂ at spectral region 4.25-14.99 μm, NaCl at spectral region 0.25-16 μm, KCl at spectral region 0.21-20 μm.

Skin as a Protective Barrier for Electromagnetic Transmission in the 9-11 μm Range

Electromagnetic radiation with a wavelength between 380 nm and 760 nm (790-400 terahertz) is detected by the human eye and perceived as visible light. Near-infrared, is from 120 to 400 THz (2,500 to 750 nm) and possess physical processes that are relevant for this range are similar to those for visible light. Mid-infrared, is from 30 THz to 120 THz (10 to 2.5 μm) are hot objects (black-body radiators) that can radiate strongly in this range. It is absorbed by molecular vibrations, where the different atoms in a molecule vibrate around their equilibrium positions. Far-infrared, is from 300 GHz (1 mm) to 30 THz (10 μm) and is in the lower part of this range and may also be called microwaves. This radiation is typically absorbed by so-called rotational modes in gas-phase molecules, by molecular motions in liquids, and by phonons in solids. The water in the Earth's atmosphere absorbs so strongly in this range that it renders the atmosphere effectively opaque. The comparison of the wavelength below, shows the spectrum of human skin and blood bandwidth. Hemoglobin exhibits strong absorption of electromagnetic waves in the visible region of 380-760 nm. The skin transmission for electromagnetic radiation has a maximum at 800-1200 nm (0.8-1.2 μm) near-infrared. The wavelength ˜950 nm (0.95 μm) is a peak maximum transmission for human skin. The depth of penetration at this point is ˜70 mm. The skin transmission for electromagnetic radiation progressively decreases above wavelength 1200 nm (1.2 μm). The region between mid-infrared and far-infrared is characterizing by strong absorption by water. The wavelength in the 9-11 μm range does not penetrate deeper at the skin as much as at superficial regions about 20-50 μm. The skin (especially water) becomes a two-way protective barrier for this range of wavelength that helps keep energy balance inside the body. Also protects against uncontrolled runaway energy in 9-11 μm range (e.g. severe burn of skin). The human aorta wall also has similar characteristic to this mid-infrared radiation.

Luminescence (Spontaneous Emission)

Luminescence is the emission of light, which is not caused by heating. Luminescence is a collective term for different phenomena where a substance emits light without being strongly heated, i.e., the emission is simply not thermal radiation. [5] This definition is also reflected by the term “cold light”. Important kinds of luminescence are fluorescence, phosphorescence, electroluminescence; and especially thermoluminescence which is a type of phosphorescence that occurs at elevated temperatures. Thermoluminescence is not to be confused with thermal radiation because the thermal excitation only triggers the release of energy from another source. Chemiluminescence is light emitted during (cold) chemical reactions. Bioluminescence is a characteristic chemiluminescence from living organisms.

The Thresholdless Emission

The thresholdless emission operates evenly below the laser threshold, the gain medium emits luminescence (for optically pumped lasers, this is called fluorescence). Above threshold (in continuous-wave operation), the intensity level of the luminescence is typically clamped to values close to that of the laser threshold (gain clamping). Just below threshold, a laser already emits some power which results from amplified spontaneous emission and has a bandwidth which is large compared to the above threshold laser emission, but is small compared to the regular luminescence bandwidth. [5] The fundamental origin of the laser threshold is the power loss via luminescence into many spatial modes (propagating in all directions). Under certain circumstances, it is possible to obtain a tresholdless laser by suppressing the luminescence within a microcavity. [11] The fundamental difference between a conventional optical cavity and microcavities is the effects that arise from the small dimensions of the system. A thresholdless laser is a laser where the threshold pump power is essentially zero and a closed microcavity is surrounded by a perfectly conductive wall. Such a device was proposed by Kobayashi in 1982. [9] An essential point is that the spontaneous emission is forced to occur primarily into the spatial mode defined by the laser resonator. When all spontaneously emitted photons are confined in a cavity whose dimensions are on the order of a single wavelength, loss to the free space mode is eliminated. [10][11] This is possible with a microcavity around the (microscopically small) gain medium, which modifies the mode structure of the environment of the gain medium. Even if there are several suitable modes, emission into the laser mode can be dominating if that mode has the highest Q factor. The Q factor is a measure of damping the resonator modes and characterizes a resonator's bandwidth relative to its center frequency. [5] Experimentally, some lasers with very low threshold powers, based on this principle, have been demonstrated, e.g., a Vertical Cavity Surface-Emitting Laser (VCSEL) with a threshold current of only 36 μW and a photonic crystal nanolaser with a threshold pump power of 1.2 μW. Another technical approach is use of the single-atom laser, which was demonstrated in 2003, and exhibits a zero threshold pump power. In this case, even more subtle quantum optics phenomena are behind the thresholdless behavior.

Distributed Bragg Reflector (DBR)

A Distributed Bragg Reflector (DBR) is a structure which consists of an alternating sequence of layers of two different optical materials, or is formed from multiple layers of alternating materials with varying refractive index, or by periodic variation of some characteristic (such as height) of a dielectric waveguide, resulting in periodic variation in the effective refractive index in the guide. Each layer boundary causes a partial reflection of an optical wave. [12] For waves whose wavelength is close to four times the optical thickness of the layers, the many reflections combine with constructive interference, and the layers act as a high-quality reflector. [5]

Electricity Background: Thermoelectric Effect

The thermoelectric effect is the direct conversion of temperature differences to electric voltage and contrariwise. [31] A thermoelectric device creates a voltage when there is a different temperature on each side creating a temperature difference between the junctions. Even if several junctions have the same temperature, and one has different, the thermoelectric effect occurs in the complete loop. This causes a continuous current in the conductors if they form a complete loop. The voltage created is of the order of several microvolts per Kelvin difference (μV/K). At atomic scale, an applied temperature gradient causes the charged carriers in the material, whether they are electrons or electron holes, to diffuse from the hot side to the cold side, similar to a classical gas that expands when heated; producing thermally induced current. [30] Charge carriers in the materials, (electrons in metals, electrons and holes in semiconductors, and ions in ionic conductors) will diffuse when one end of a conductor is at a different temperature than the other. Hot carriers diffuse from the hot end to the cold end, since there is a lower density of hot carriers at the cold end of the conductor. Cold carriers diffuse from the cold end to the hot end for the same reason. If the conductors were left to reach thermodynamic equilibrium, this process would result in heat being distributed evenly throughout the conductor. The movement of heat in the form of hot charge carriers from one end to the other is called a heat current. As moving charge carriers, it is also as an electric current. If the rate of diffusion of hot and cold carriers in opposite directions is equal, there is no net change in charge.

Deep Brain Stimulation (DBS)

The method of deep brain stimulation (DBS) consists of implantation of tiny microelectrodes in to the brain to deliver stimulation pulses to the tissue by electrical pulse generator (PG) e.g., 100 Hz, 10 V, 90 μs-widths, cycling 1 min/on, 5 min/off. In spite of a many studies using DBS, its mechanism of action is still not well understood. Developing DBS microelectrodes is still challenging. [21]

### REFERENCES

1. Lumb A B Nunn's Applied Respiratory Physiology 2005, 501 pages; 6^{th} edition Elsevier Ltd; ISBN 0750687916
2. Kikuchi R, Kikuchi K, Hildebrandt J, Yanai M, Sekizawa K, Sasaki H. “Dependence of collateral and small airway resistances of CO₂ and volume in dog lobes.” Respir Physiol. 1995 June; 100(3):245-52.
3. Martin L D, Krunkosky T M, Dye J A, Fischer B M, Jiang N F, Rochelle L G, Akley N J, Dreher K L, and Adler K B. “The Role of Reactive Oxygen and Nitrogen Species in the Response of Airway Epithelium to Particulates.” Environmental Health Perspectives—Vol 105, Supplement 5, September 1997
4. Fink M P. “Role of reactive oxygen and nitrogen species in acute respiratory distress syndrome.” Curr Opin Crit Care. 2002, February, 8(1):6-11
5. Paschotta. R “Encyclopedia of Laser Physics and Technology.” 2008. XII, 844 Pages, 2 Volumes, Wiley-VCH, Berlin ISBN-10: 3-527-40828-2, ISBN-13: 978-3-527-40828-3
6. Krishna C M, Bose B, Gupta P K. N₂ laser-induced oxidation of hemoproteins in red blood cell lysate. Radiat Res 2000; 153: 411-415.
7. Kwasny M, Nowicki K, Strzelec M, Jakubowski J. “The influence of weak CO₂ laser radiation on the blood parameters.” Optoelectronica 4, March 1996.
8. Wasik M, Gorska E, Modzelewska M, Nowicki K, Jakubczak B, Demkow U “The influence of low-power helium-neon laser irradiation on function of selected peripheral blood cells.” J Physiol Pharmacol. 2007 November; 58 Suppl 5(Pt 2):729-3
9. Kobayashi T, Segawa T, Morimoto Y. Sueta T, “Novel-type lasers, emitting devices, and functional optical devices by controlling spontaneous emission”, presented at the 46th Fall Meeting of the Japanese Applied Physics Society, 1982, paper 29a-B-6 (in Japanese)
10. Protsenko I, Domokos P, Lefevre-Seguin V. et al., “Quantum theory of a thresholdless laser”, Phys. Rev. A 59 (2), 1667 (1999)
11. Yokoyama H., “Physics and Device applications of optical microcavities” Science, New Series 3 April 1992, Vol 256, No 5053, 66-70
12. Baba T, Hamano T, Koyama F, Iga K, “Spontaneous Emission Factor of Microcavity DBR Surface-Emitting Laser” Journal of Quantum Electronics 27, (6), June 1991, 1347-1358
13. Griffiths P R de Haseth J A “Fourier Transform Infrared Spectroscopy” 2007 Wiley, 2^{nd} ed., ISBN 978-0-471-19404-0
14. El-Bermani A W, Grant M, “Acetylcholinesterase-positive nerves of the rhesus monkey bronchial tree”, Thorax 1975; 30:162-170 doi:10.1136/thx.30.2.162
15. Shawnessy D. Larson S D, Schelegle E S, Hyde D M, and Plopper C G. “The Three-Dimensional Distribution of Nerves along the Entire Intrapulmonary Airway Tree of the Adult Rat and the Anatomical Relationship between Nerves and Neuroepithelial Bodies” American Journal of Respiratory Cell and Molecular Biology 2003, 28: 592-599.
16. Conboy I M, Conboy M J, Wagers A J, Girmal E R, Weissman I L & Rando T A “Rejuvenation of aged progenitor cells by exposure to a young systemic environment” Nature, 17 Feb. 2005, 433: 760-764.
17. Raju S, Heath D B J, Warren E T, and Hardy J D “Single- and Double-lung Transplantation Problems and Possible Solutions” Ann. Surg. June 1990 Vol. 211 No. 6: 681-691
18. Mahan J R. “Radiation heat transfer: a statistical approach”, Vol 1, Wiley 2002, New York, ISBN 0-471-21270-9
19. Brütting, W. Physics of organic semiconductors. 2005 Wiley-VCH. ISBN 9783527405503
20. Hirsch J “Hopping transport in disordered aromatic solids: a re-interpretation of mobility measurements on PKV and TNF”. J of Physics C: Solid State Physics (1979) 12 (2): 321
21. Kringelbach M L, Jenkinson N, Owen S L, Aziz T Z (August 2007). “Translational principles of deep brain stimulation”. Nat. Rev. Neurosci. 8 (8): 623-35. PMID 17637800
22. Ko D, Heck C, Grafton S, et all “Vagus Nerve Stimulation Activates Central Nervous System Structures in Epileptic Patients During PET H₂¹⁵O Blood Flow Imaging” Neurosurgery, August 1996, 39(2): 426-431.
23. Gumustekin K, Seven B, Karabulut N, Aktas O, Gursan N, Aslan S, Keles M, Varoglu E, Dane S, “Effects of sleep deprivation, nicotine, and selenium on wound healing in rats” (Abstract). Int J Neurosci 2004; 114(11): 1433-42.
24. Zager A, Andersen M. L, Ruiz F S, Antunes I B, & Tufik S, (2007). “Effects of acute and chronic sleep loss on immune modulation of rats” [Electronic version]. Regulatory, Integrative and Comparative Physiology, 293, R504-R509.
25. Silber M H, Ancoli-Israel S, Bonnet M H, et al. (March 2007). “The visual scoring of sleep in adults”. J Cl Sleep Med. 3 (2); 121-31. PMID 17557422.
26. Engel G S, Calhoun T R, Read E L, et all. “Evidence for wavelike energy transfer through quantum coherence in photosynthetic systems”, 2007, Nature 446, 782-786 (12 Apr. 2007)
27. Scholes G D (7 Jan. 2010), “Quantum-coherent electronic energy transfer: Did Nature think of it first?”, J of Phys Chem Lett 1, 1: 2-8, doi:10.1021/jz900062f
28. Collini E, Wong C Y, Wilk K E, Scholes G D et all. “Coherently wired light-harvesting in photosynthetic marine algae at ambient temperature”, 2010 Nature 463 (7281), Bibcode 2010 Natur. 463. 644C, doi:10.1038/nature08811, PMID 20130647
29. Campbell, N A. Reece J B, (2005). Biology, 7th Edition. San Francisco: Pearson-Benjamin Cummings. pp. 186-191. ISBN 0-8053-7171-0.
30. Rowe, D. M., (2006)“Thermoelectrics Handbook: Macro to Nano” Taylor & Francis. ISBN 0-8493-2264-2.
31. Besancon, Robert M. (1985). “The Encyclopedia of Physics, Third Edition” Van Nostrand Reinhold Company ISBN 0-442-25778-3.
32. Planel E, Richter K E, Nolan C E, Finley J E, Liu L, Wen Y, Krishnamurthy P, Herman M, Wang L, Schachter J B, Nelson R B, Lau L F, Duff K E. “Anesthesia leads to tau hyperphosphorylation through inhibition of phosphatase activity by hypothermia” J Neurosci. 2007 Mar. 21; 27(12):3090-7
33. Planel E, Bretteville A, Liu L, Virag L, Du A L, Yu W H, Dickson D W, Whittington R A, Duff K E “Acceleration and persistence of neurofibrillary pathology in a mouse model of tauopathy following anesthesia” FASEB J. 2009 August; 23(8):2595-604. Epub 2009 Mar. 11
34. Run X, Liang Z, Zhang L, Iqbal K, Grundke-Iqbal I, Gong C X. “Anesthesia induces phosphorylation of tau” J Alzheimers Dis. 2009 March; 16(3): 619-626.
35. Kelly G. (2006) “Body temperature variability (Part 1): a review of the history of body temperature and its variability due to site selection, biological rhythms, fitness, and aging” Altern Med Rev. December; 11(4):278-93
36. Kelly G. (2007) “Body temperature variability (Part 2): masking influences of body temperature variability and a review of body temperature variability in disease” Altern Med Rev. March; 12(1):49-62.
37. Curran L K, Newschaffer C J, Lee L C, Crawford S O, Johnston M V, Zimmerman A W. (2007) Behaviors associated with fever in children with autism spectrum disorders.

Pediatrics 2007; 120:e1386-92. [PubMed: 18055656]

### BRIEF SUMMARY OF THE INVENTION

The modern respiratory physiology is based on passive oxygen diffusion, and the new model of respiratory physiology is based on the water dissociation catalyzed by exited hemoglobin as organic semiconductor at low-power density radiation ˜0.1 μW per unit emitted from excited CO₂ molecules. The Terminal and Respiratory Bronchioles System (TRBS), is responsible for this process. TRBS is a composite of three modules: (a) The Tunable Microcavity Module—that is in the terminal bronchioles, (b) The Compressor Module—that is in the respiratory bronchioles and alveolar ducts, (c) The Reservoir Module—that is in the alveolar sacs. Two types of optical processes lead to the energy amplification: Spontaneous Emission and Thresholdless Emission in a Tunable Microcavity. The collateral ventilation system is responsible for CO₂ distribution. The Tunable Microcavity is covered by a surfactant that acts as a multilayer reflective surface and corresponds to Distributed Bragg Reflector (the DBR). The vagal nerve innervates the microcavity and supervises the muscles contraction (Q factor). The compressors (smooth muscles of respiratory bronchioles and alveolar ducts) control the CO₂ cooling process. The collisions of the colder CO₂ molecules with the warmer homonuclear N₂ molecules, excites to a vibrational metastable level leads to the population inversion via vibrational excitation of the CO₂ necessary for lasing operation in microcavity. The vibrational modes lattice transport energy to cells and genome via the blood circulatory system with oxygen. Significantly, the effectiveness of the energy transfers by vibrational modes is very high in a living organism. In the mid-infrared range a minimal energy excitation of molecule to a higher vibration state that is less than 0.5 eV. The energy difference for transitions between the ground state (υᵢ=0) and the first excited state (υᵢ=1) of most vibrational modes corresponds to the energy of radiation in the mid-infrared spectrum 25 μm-2.5 μm (400-4000 cm⁻¹). The mostly applied CO₂ molecules bandwidth is at a 9-11 μm with peaks 9.6 μm and 10.6 μm. Based on the principles of Debye model of solid body, Planck's law, Wien's Displacement Law and infrared and Raman spectroscopy the TRBS can be replaced or improved only by use of a tunable CO₂ low power laser that is a substitute of a life sustaining source of energy. Some diseases can be explained as having a deficit of coherent energy [e.g., infant sudden death—is an example of a sudden energy deficit, Autism Spectrum Disorder (ASD)—is a partial deficit that occurs during developmental onset, Alzheimer disease—is a severe deficit seen in aging, Acquired Immune Deficiency Syndrome (AIDS), —is an acquired deficit.] Significant deficit of the infrared energy in CO₂ bandwidth is cause of death a mammalian organism regardless of mechanism leading to deficit. All these disorders have the potential to be treated with optoelectronics.

### BRIEF DESCRIPTION OF THE SEVERAL VIEWS OF THE DRAWING

FIG. 1

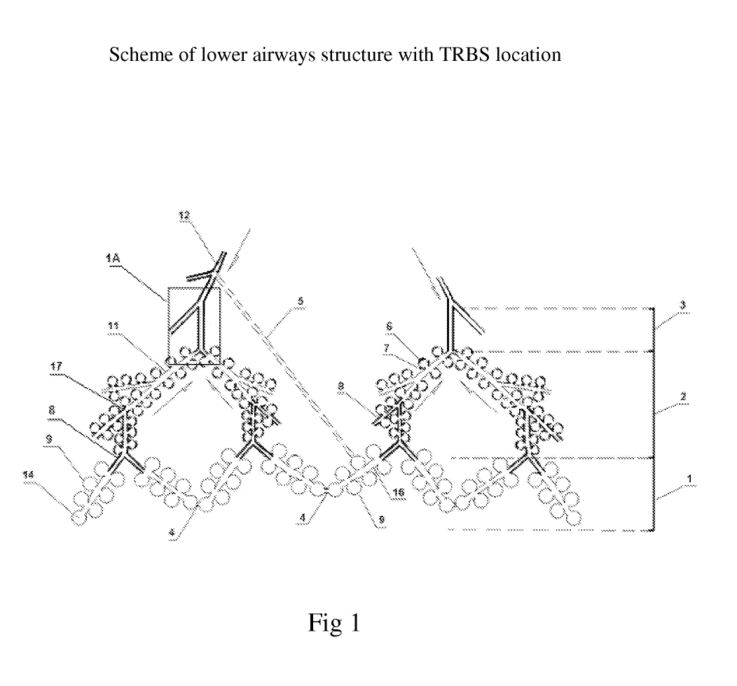

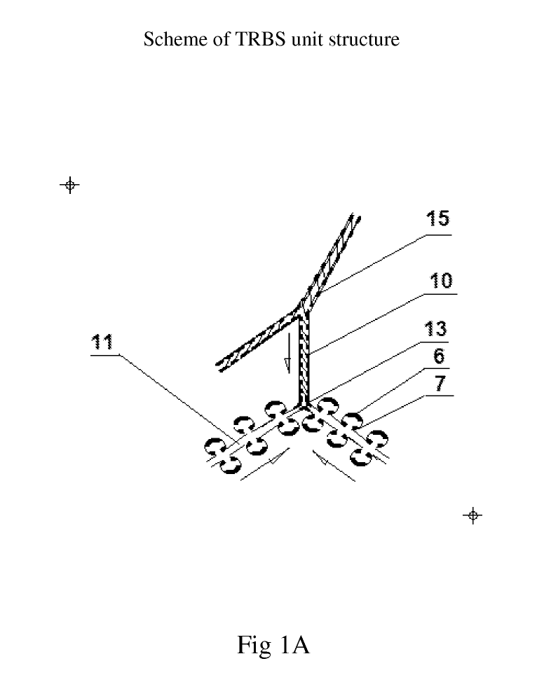

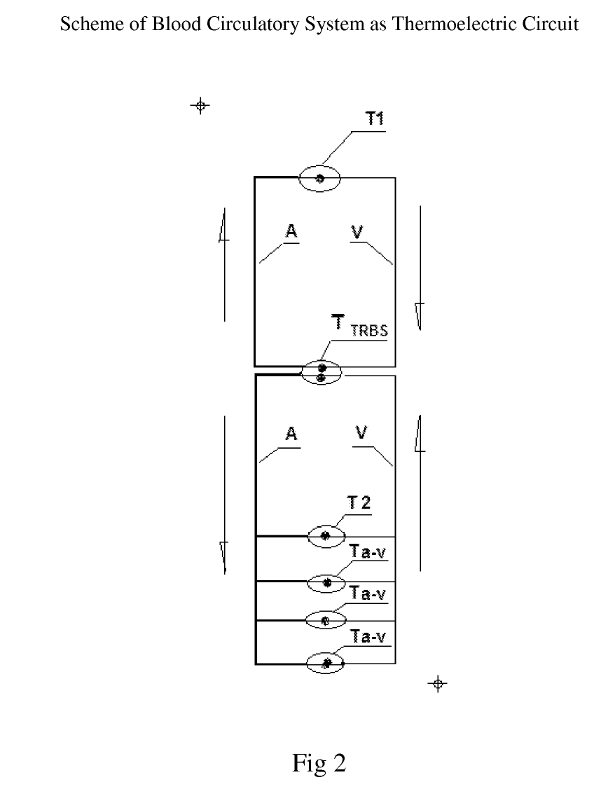

Scheme of lower airways structure with TRBS location
1a Link to the magnification of TRBS unit (FIG. 1a)
1. Reservoir module—the alveolar sacs
2. Compressor module—the alveolar ducts, respiratory bronchioles
3. Microcavity module—the terminal bronchioles
4. Alveolar pores (pores of Kohn)
5. Canals of Lambert (Lambert's sinuses)
6. The smooth muscle layer on the mural alveoli of the respiratory bronchiole
7. The smooth muscle layer with band that loop over the mouths of the mural alveoli of respiratory bronchiole
8. Alveolar ducts—alveolar septa comprise a series of rings forming the walls and containing smooth muscle.
9. The alveolus
11. Respiratory bronchiole generation 15
12. Terminal bronchiole generation 12
14. Lumen of the alveolus with CO₂
16. Alveolar sacs generation 23
17. The bands of smooth muscle that loop over the opening of the alveolar ducts to respiratory bronchiole
↓ Direction of the air flow (O₂, N₂, CO₂, ROS, RNS, H₂O vapors) during inhalation
↑ Direction of the carbon dioxide flow with compression during inhalation and exhalation through the respiratory bronchioles

FIG. 1a

Scheme of TRBS unit structure
6. The smooth muscle layer on the mural alveoli of the respiratory bronchiole
7. The smooth muscle layer with band that loop over the mouths of the mural alveoli of respiratory bronchiole
11. Respiratory bronchiole generation 15
13. Nozzle between respiratory and terminale bronchioles
10. Terminal bronchiole generation 14
15. Strong helical muscular bands of terminal bronchioles
↓ Direction of the air flow (O₂, N₂, CO₂, ROS, RNS, H₂O vapors) during inhalation
↑ Direction of the carbon dioxide flow with compression during inhalation and exhalation through the respiratory bronchioles

FIG. 2

Scheme of Blood Circulatory System as Thermoelectric Circuit

T_{TRBS}>Tn

T—Temperature

Tn—Temperature of T1, T2, T a-v

T_{TRBS}—Temperature of TRBS

T1—Temperature of brain's arterial-venous junctions

T2—Temperature of heart's arterial-venous junctions

T a-v—Temperature of random arterial-venous junction

A—Artery

V—Vein

↑↓—Blood flow and energy flow directions

### DETAILED DESCRIPTION OF TERMINAL AND RESPIRATORY BRONCHIOLES SYSTEM (TRBS)

The TRB system is composed of three modules (FIG. 1):

1. Tunable microcavity module—in the terminal bronchioles

2. Compressor module—in the alveolar ducts and respiratory bronchioles

3. Reservoir module—in the alveolar sacs

Anatomically, the TRBS intake zones of adult lungs are the pulmonary acinus (syn. primary lobule, terminal respiratory unit) generations 15^{th}-23^{th} and terminal bronchioles generation 12^{th}-14^{th}. The pulmonary acinus represents eight generations (generations 15^{th}-23^{th}), but in practice the number of generations within a single acinus is variable between six and twelve divisions beyond the terminal bronchiole. Assumed that a middle point of the TRBS unit is the last generations (the 14^{th}) of the terminal bronchioles and the first generation (the 15^{th}) of the respiratory bronchioles, the number of TRBS units is ˜1.6·10⁴ per one acinus. A human lung contains about 3.0·10⁴ acini. This makes the TRBS units about 4.8·10⁸ in a single lung and, ˜9.6·10⁸ in both lungs. The middle point of the TRBS unit base is located as a “Y” shape junction composed of two ends of the respiratory bronchioles and one end of the terminal bronchiole (FIG. 1a.). On both sides of the middle point of TRBS unit the three modules are located: reservoir module, corresponding to the alveolar sacs connecting to the alveolar ducts, compressor modules corresponding to the alveolar ducts and respiratory bronchioles, and the microcavity module corresponding to the terminal bronchioles on opposite side of middle point. The alveolar sacs are forming the reservoir for CO₂. The interalveolar pores are responsible for CO₂ equal distribution and bronchioloalveolar channels of collateral ventilation purging of CO₂ surplus to adjacent terminal or respiratory bronchioles for enrich gases mixture. The CO₂ is a main source of mid-infrared radiation for TRBS. The alveolar sacs have an important, peripheral location in the lung. During inhalation the peripheral zone of lungs is pressed from both sides: by chest wall, and inspired air under ambient pressure. It works like a pump that is pumping CO₂ from alveolar sacs to respiratory bronchioles via the alveolar ducts. During exhalation, the arterial pCO₂ does not significantly fall; however pCO₂ rises in the alveoli lumen, the differences are mostly significant during the effort, when influent on breathing control. The alveolar septa comprise a series of muscular rings forming the walls of the alveolar ducts. The mean diameter of the alveolar ducts is 0.3 mm. About half of the alveoli rise from the ducts and approximately 35% of alveolar gases reside in the alveolar ducts and the alveoli that arise directly from them. The smooth muscle rings functioning as the valves blocking the CO₂ returning to alveoli when the next portion of CO₂ is accumulated from the pulmonary capillaries. Every respiratory bronchiole (generation 15^{th}-18^{th}) is connected to two alveolar ducts (generation 19^{th}-22^{th}). The mean diameter of the respiratory bronchioles is 0.4 mm. The respiratory bronchioles (generations 15^{th}-18^{th}), are embedded in lung parenchyma; however, they have a well-defined muscle layer with a band that loops over the opening of the alveolar ducts and the mouth of the mural alveoli so, that the alveolar duct and respiratory bronchiole have a wall surrounded by smooth muscle. The smooth muscle bands of respiratory bronchioles and alveolar ducts play a part in CO₂ compressor. The CO₂ is warms up progressively (adiabatic process) when it is pressed by the smooth muscles within the alveolar ducts and respiratory bronchioles passage and by fast decompression is cooling and injected to terminal bronchioles microcavity. In practice, the number of the respiratory bronchioles beyond the terminal bronchiole is quite variable. Respectively, this can significantly improve or reduce the compressor effectiveness. The terminal bronchioles (generations 12^{th}-14^{th}) are located on the opposite side of the midpoint of the TRBS unit. The terminal bronchioles are the last generations which conduct inhaled air. The diameter of the terminal bronchioles close to the midpoint is 0.4 mm; however, the mean diameter is 0.7 mm. Cartilage disappears from the wall below the 11^{th} generation and ceases to be a factor in maintaining patency. It is extremely important to form the microcavity. The strong helical smooth muscles bands appearing in the terminal bronchioles walls providing resistance to the bronchioles which can increase to very high values when there are contracted for protect and support of the TRBS function. The most important part of TRBS is connectivity with central nerves system (CNS). The medulla is the area of the brain where the respiratory pattern is generated and where gas exchange is coordinated. The medullary chemoreceptors monitor a rise in arterial P_{CO2} and depend on the TRBS requirements, respiratory depth and rate increase until a steady state of hyperventilation is achieved. About 85% of the total respiratory response to inhaled CO₂ originates in these medullary chemoreceptors. According to the presence of a microcavity, the parasympathetic system is more important than sympathetic, which is poorly represented in the human lung. Both afferent and efferent fibers travel to the lung in the vagus nerve with efferent ganglia in the wall of small bronchi. The parasympathetic system is responsible for bronchomotor tone which controls the Q factor of the tunable microcavity. The nerve plexuses extend as far as the respiratory bronchioles and control the compressor function. In the bronchioles, the nerves in the mucosal fold are close to the mucosal surface. In the bronchial submucosa, there are acetylcholinesterase-positive (AChE-positive) nerve plexuses which arise from three sources: (1) the adventitial plexus in bronchioles or the subchondral plexus in bronchi; (2) the perimuscular nerve plexus, and (3) AChE-containing nerves associated with the bronchial artery. The submucosal plexus appears to innervate the acinar submucosal glands in the bronchi as well as continuing as central nerves in the mucosal folds. The airways of some animals and healthy humans can be tonically constricted, and this tone is maintained by vagal efferent nervous activity. Significantly the tonic constriction from the terminals bronchioles and alveolar ducts are unaffected by reflex bronchoconstriction, so the lung compliance is usually unchanged unless the bronchoconstriction is severe. This is proof for the unique, life sustaining innervation of TRBS that exists by vagal nerve fibers, ganglions cells and nerve plexuses. Vagal nerve stimulation leads to activate the anterior thalamus (part of limbic system), posterior temporal cortex, putamen and inferior cerebellum. These connections are influents on the energy production by the TRBS via innervation of tunable microcavity module i.e., Q factor control via the helical smooth muscles in terminal bronchioles, and compressor module i.e., CO₂ cooling control via the smooth muscles in respiratory bronchioles. The uncontrolled power density changes of TRBS (e.g., energy falls) can significantly harm some central areas and lead to malfunction (e.g., epilepsy). The severe energy fall of TRBS can lead to death (e.g., sudden infant death). The cut of vagal nerve or denervation during lungs transplantation leads to disconnection of central areas of nerves system and keeps only basic automatic function of ganglion cells and nerves plexuses along bronchi and bronchioles support TRBS function. The lost control on TRBS by central areas of CNS via vagal it is not lost of full control on Q factor in microcavity and compressor function. This leads to a lack of precisely power output responses of TRBS for inside and outside stimulus and problems with power output coordination in both lungs or lobes in a given lung. The process is related to heart automatism but it is more complex because of the three modules. The sudden lost of coordination between TRBS, heart and CNS connections via vagal nerve can lead to a temporary blood pressure disorder. Resetting of the receptors that control oxygenation, pH, and carbon dioxide tension appears but takes usually several weeks. The scheme of structure and distribution of pulmonary arteries, which lie close to the corresponding air passages in connective tissue sheaths, support a regulatory mechanism of perfusion sped by vascular resistance and TRBS energy transmission. The media of the pulmonary arteries is about half as thick as in the systemic arteries of corresponding size and carries only one-sixth of the systemic arterial pressure. In the smaller vessels, media is mainly muscular where the transition exists in vessels of about 1 mm in diameter. These types of vessels regulate the blood perfusion. The pulmonary arterioles occur in an internal diameter of about 0.1 mm and lies near the terminal bronchioles. These vessels differ radically from their counterparts in systemic circulation because they are virtually devoid of muscular tissue. There is a thin media of elastic tissue that is separated from the blood by the endothelium. These types of vessels are supports energy transmitting from TRBS. There, blood remains exposed on the emission of CO₂ bandwidth from the terminal bronchioles microcavities and the hemoglobin begins to alter to the red color of oxyhaemoglobin within the pulmonary arterioles before the blood enters the pulmonary capillaries. Furthermore, according to Henry's law the heated blood better support CO₂ diffusion from plasma to alveolar space through pulmonary capillaries which do not cross a single alveolus, but may pass over three or more. Down to the terminal bronchioles, the air passages derive its nutrition and heat from the bronchial circulation. The pulmonary veins are located far from bronchioles and lie close to the septa that separate the segments of the lung. The presence of a thin media of elastic tissue in the pulmonary arterioles wall compose without a muscular layer, and CO₂, glucose, NaCl, KCl, within the plasma are support good transmission of vibrational modes at 9.2-9.4 μm i.e., the CO₂ bandwidth via ion and molecular channels. The proper concentration of plasma components counteracts the absorption gain of mid-infrared waves by water in plasma. Two types of optical processes lead to energy amplification:

1. Spontaneous emission (luminescence)

2. Thresholdless emission in a tunable microcavity

Spontaneous emission (luminescence) progressively appears in the bronchi above the terminal bronchioles region. The temperature in the bronchi increases to ˜38° C. because the heat emission from the bronchial arteries and frictional forces during turbulent gas flow that leads to energy rise. The thermal excitation only triggers the release of energy from air molecules which occur at elevated temperatures. This is not to be confused with thermal radiation. Luminescence is the light emission which is not caused by heating. During inhalation the progressive collisions of particles transferred vibrational excitation between: reactive oxygen species and reactive nitrogen species (ROS/RNS), O₂, N₂, CO₂ within the lumen of bronchi and bronchioles lead to luminescence. In the mid-infrared range a minimal energy excitation of molecule to a higher vibration state that is less than 0.5 eV. The process contains a few steps. The airways epithelium itself is directly capable of generating ROS/RNS. The inelastic and elastic collides that occur between molecules of ROS and RNS with O₂ and N₂, during the thermal excitation and turbulent gas flows finally leads to a metastable vibrational level of the nitrogen molecules. The nitrogen is a homonuclear molecule and cannot rapidly lose this energy by photon emission, so excited vibrational levels are metastable and exist for a relatively long time in atomic scale. The remains of the CO₂ molecules appear in the airways after exhalation and the nitrogen molecules that are excited into a metastable vibrational level can transfer by collision, its vibrational excitation to the CO₂ molecules. The process leads to a population inversion by excitation into a vibrational level for the carbon dioxide molecules. The carbon dioxide releases the energy in the form of a photon that becomes the source of spontaneous emission. The luminescence characterizes a large number of spatial modes propagating in all directions, so energy absorption by the gain medium (presence of water vapors) is very strong compared to the weak emission that exist far below the lasing threshold. The terminal bronchioles became a transition region that changes spontaneous emission to a tresholdless emission. The thresholdless emission takes place in the terminal bronchioles (generation 12^{th}-14^{th}). The principle of thresholdless emission suppresses the luminescence within a microcavity. When all spontaneously emitted photons are confined in a cavity whose dimensions are on the order of a single wavelength, loss to the free space mode is eliminated. The walls form a microcavity that is covered by surfactant serves as a multilayer reflective surface that corresponds to a Distributed Bragg Reflector (DBR mirror with corrugated waveguide structures). The resonator gaps for the CO₂ bandwidth can be tunable for contraction of the smooth muscles helical bands starting at 0 μm to the size require of wavelength 9.2-9.4 μm matches with core temperature in lungs (Appendix 4). Collisional transferring of vibrational energy from warmer N₂ to the colder CO₂ takes place when the luminescence appears at the entry point of microcavity (close to midpoint of TRBS). The numerous warm ˜38° C. nitrogen molecules excited to a metastable vibrational level as they move toward the respiratory bronchioles (compressor) and concurrently the compressor generate an opposite flux of colder CO₂ that collides with the excited nitrogen molecules. Collisional energy transfer between the excited nitrogen and the carbon dioxide molecule causes vibrational excitation of the carbon dioxide, with sufficient efficiency to lead to the population inversion that is necessary for lasing operation. Water vapor helps stabilize the process in equilibrium at the operating temperature. The direction of the wave's propagation is mainly perpendicular to longitudinal axis of microcavity. Essentially, spontaneous emission is forced to occur primarily into the spatial mode defined by the laser resonator. The limited resistance of the organic resonator structure leads to decrease a wavelength than to increase a wave's amplitude for multiplies the power output. The nerve receptors in the mucosal folds detect energy change. The helical smooth muscle bands start reaching the highest Q factor of modes which appeared in the microcavity. If there are several suitable modes, emission into the laser mode can be dominating if that mode has the highest Q factor. The process leads to a low power output of 0.1 μW per TRBS unit [Appendix 1]. The Q factor is under control of the ganglion cells and nervous plexuses linked to the vagal parasympathetic AChE-positive fibers. The vagal nerve is connected to the anterior thalamus (part of the limbic system), the posterior temporal cortex, putamen and inferior cerebellum. The central nervous system (CNS) controls the TRBS power output via the Q factor and compressor effectiveness. The cooled CO₂ processed in the compressor module is around room temperature (it depends on the compressor effectiveness) and helps keep reabsorption at the lower level. This can be compared to a Quasi-Three-Level System where the lower laser level is very close to the ground state that is an appreciable population that occurs in thermal equilibrium at the operating temperature. As a consequence, the unpumped gain medium causes some reabsorption loss at the laser wavelength and transparency is only reached for some finite pump intensity. In the case of TRBS, the thresholdless emission in a tunable microcavity can be reached without threshold pump power. The circulation of gases within the terminal bronchioles is maintained by the inhalation and exhalation cycle. Adding more oxygen to improve thresholdless emission gives a moderate output limited by an optimal gases ratio in the microcavity. In practice, the higher oxygen changes in ventilation above the normal level have comparatively little effect on arterial pO₂. In contrast, changes in ventilation below the normal level may have a very marked effect. At very low levels of ventilation, the arterial pO₂ becomes critical and small changes may precipitate severe hypoxia. The surfactant antioxidant properties protect the bronchioles wall against harm by reactive species. The surfactant monolayer and multilayer phospholipids covered microcavity alternates between two forms because of the SP-B and SP-C proteins. This property is support “leak” of coherent waves through the surfactant layers, and rapid conversion from photons energy of coherent waves to vibrational excitation of bonds, groups and molecules on the same resonant frequency and same phase in the wall of the microcavity and the tiny wall of pulmonary arteriole. The normal modes can move independently (multimodal system), and excitation of one mode will never cause motion of a different mode. The photons energy of coherent wave can be converts to a vibrational mode on the same frequency and phase. This mean that the effectiveness of the energy transfers by vibrational modes (wavelike energy transfer) is very high in a living organism. The energy difference for transitions between the ground state (υᵢ=0) and the first excited state (υᵢ=1) of most vibrational modes corresponds to the energy of radiation in the mid-infrared spectrum 25 μm-2.5 μm (400-4000 cm⁻¹). In the mid-infrared range a minimal energy excitation of molecule to a higher vibration state is less than 0.5 eV. Vibrational modes in resonant frequency of the CO₂ bandwidth appear within the pulmonary arteriole. The excitation of hemoglobin molecules processes via plasma molecular channels i.e., CO₂, glucose, NaCl, KCl. The organic semiconductors characterized optical properties such as absorption and photoluminescence. The hemoglobin as organic semiconductor excitation consist of absorbs of wavelike energy transfer by hem (pigment) in the mid-infrared long interaction times of the hemoglobin molecule with the radiation field and without the possibility for rapid cooling. The carbamino carriage by hemoglobin inside the red blood cell helps infrared energy transfers to heme groups in CO₂ resonance bandwidth. The absorbed quantum coherence energy causes the formation of an exciton (an electron excited to a higher energy state) in the pigment molecule. The mechanism of highest occupied molecular orbital and lowest unoccupied molecular orbital gap (HOMO and LUMO gap) support the energy flow and quantum mechanical tunneling in organic semiconductors like hemoglobin and myoglobin. Simply, the quantum tunneling allows the electrons to go through the potential barrier (e.g., 13.8 eV for CO₂, 13.6 eV for H or O electrons) instead of going all the way over it because of the wave nature of the electron. The electron-deficient hemoglobin become an oxidizing agent. The electronic properties of organic semiconductors such as ionization energy (E_{I}) of molecule means the energy needed to remove electrons from atom. The water-splitting reaction is catalyzed by the electron-deficient hemoglobin as an oxidizing agent which allows to break apart molecules as stable as water (i.e., quasi photoelectrochemical cell properties called “artificial photosynthesis” where photo-anode made of the n-type semiconductor and the cathode made of a metal). The photo-vibrational dissociation leads to increases the oxyhemoglobin compounds. The surplus of energy at the CO₂ bandwidth is propagates within plasma by molecular channels i.e., CO₂ (spectral range 4.25-14.99 μm of wavelength), NaCl (spectral range 0.25-16 μm of wavelength), KCl (spectral range 0.21-20 μm of wavelength), and glucose (spectral range 8.5-10 μm of wavelength), transferring via a vibrational modes lattice to the body cells and neurons. An especially the CO₂ properties are manifested as easy diffusion through the membranes of all cells in human body. The basic scheme of energy distribution via the circulatory blood system matches with the thermoelectric closed loops that have many “colder junctions” and one “warm junction” which is the TRBS in lungs. [FIG. 2] The heart pumps blood with many ions, gas molecules, protein compounds and cells throughout the body; however the propagation of vibrational modes at the CO₂ bandwidth causes small temperature difference (μV/K) between TRBS and arterial-venous capillary junctions. The vibrational mode causes energy diffuses and powers the body cells because infrared waves are the fraction of wide spectrum of electromagnetic waves family. If the circulatory system were left to reach thermodynamic equilibrium, this process would result in energy being distributed evenly throughout the circulatory system. Significantly, the brain has a relatively shorter thermoelectric loop because greater electromagnetic energy that is consumed and the shorter delivery times are needed. The heart has the shortest loop and energy consumption is related to its load. The most important problem to address is the CO₂ distribution to TRBS. In the human body there is feedback between energy emission by TRBS and CO₂ delivery from tissues and appears as follows in Appendix 3. The oxyhemoglobin dissociation curve (The Bohr Effect) is shifted to the right when the temperature rises high, pH is lower and pCO₂ is increased, this leads to an increase of CO₂ delivery to TRBS and a higher energy production. It is a general rule that a shift to the right (increased P₅₀) will benefit the venous pO₂, providing that the arterial pO₂ is not critically reduced. The shift to the left corresponds to a lower than normal temperature, higher pH, and decrease of pCO₂ that leads to a decrease “fuel” delivery to TRBS where the power density of coherent waves falls. In the brain, the medulla is responsible for maintaining the balance between the CO₂ delivery and TRBS energy emission in a resting state or during effort. The medullary chemoreceptors monitor a rise in arterial P_{CO2}, respiratory depth and the rate increases until a steady state of hyperventilation is achieved. About 85% of the total respiratory response to inhaled CO₂ originates in these medullary chemoreceptors. Partial oxygen pressure is monitoring by the chemoreceptors that are located close to the bifurcation of the common carotid artery, which are sensitive to a fall in pO₂ levels. The TRBS is a resistant, dynamic neuromuscular system; however the smooth muscle cells which build the moving parts must be regenerated during a 24 hours cycle. Sleep is an irreplaceable component of TRBS that is responsible for the metabolic restoration of the neuromuscular components directly linked to a power output level. Lack of the sleep drives to coherent waves emission decline in the mid-infrared bandwidth with significant changes in the immune system, neurological consequences and malfunction of internal organs that leads to death in an extreme cases (with lost of powering neurons). The deepness of sleep and it phases are depends on a number of “disconnected-relaxed” TRBS units where energy falls exhibits with a slightly lower temperature. The TRBS mid-infrared energy in the 9.2-9.4 μm bandwidth appeared as an unrecognized common factor of many physiological processes. The arterial P_{O2} shows a progressive decrease with age exhibited with the Marshall-Whyche's equation that describes a decrease of TRBS sufficiency with age. In animal models the results of experiments with muscle regeneration in young 2-3 month old transgenic mice, and aged 19-26 month old transgenic mice, in heterochronic and isochronic pairings suggest that the age-related decline of progenitor cell activity can be modulated by systemic factors that change with age. It is matching to decrease of mammalian TRBS sufficiency with age. The method of Deep Brain Stimulation (DBS), consists in implantation of tiny microelectrodes into the brain to deliver stimulation pulses to the tissue by electrical pulse generator that are similar to the thermoelectric loops properties (common electromagnetic stimulus and different frequency) powered the body cells and neurons via the blood circulatory system. The problem of atelectasis and an associated increased ˜10% venous admixture during anaesthesia is related to the TRBS compressor impaired. After anaesthetic agents is used the opposite flux of colder CO₂ from compressor declines because the fast purging of carbon dioxide leads to decreases CO₂ pressure in alveolar ducts and respiratory bronchioles and only fresh air administrated by anaesthesist flowing to TRBS units in accordance to oxygen passive diffusion theory. The anaesthetics block the compressor smooth muscles function and CO₂ diffusion result in dysfunction that leads to Acute Hypoxic Ventilatory Response (AHVR), which consists in impairs the TRBS quantum coherent waves production and appears as hypercapnia and hypoxia. The hypercapnia is an extra CO₂ release from tissues as a response to decline of the CO₂ energetic effectiveness which resulted in hypoxia. General anesthesia associated with increased risk of Alzheimer's disease, because anesthesia induces hypothermia, which leads to overt tau hyperphosphorylation in the brain of regardless of the anesthetic used. It is linked to decline of mid-infrared energy production by TRBS (compressor impairs) during general anesthesia and manifests by temperature decreased. The nerves system supports a live sustaining protection of TRBS units by smooth muscle contraction at the bronchi level to minimize consequences of inhaled anaesthetics (˜90% of alveolar volume do not containing atelectasis). The respond is known as the vagal reflex that increases airway and tissue resistance, following a vagal stimulation. In anesthesia using an artificial ventilation unit, routine monitoring of end-expiratory pCO₂ helps adjust the target pCO₂ selected by the anaesthetist. The lack of TRBS compressor function in deep anaesthesia drives to apnoea because the anaesthetic agents reduce the pCO₂/ventilation response and there may be no response below the pCO₂ apneic threshold. The CO₂ that is added to the inspired gas improves a weak spontaneous emission in the microcavities (without a cooler flux of the CO₂ from the compressor). The lack of a reverse mechanism in the compressor function results in the TRBS general malfunction and the subject's death. Reverse of the energy deficit, which appears during aberration of physiological process makes it possible for apply a low power, tunable CO₂ laser in 9.2-9.4 μm bandwidth based on principles of Debye model of solid body, Planck's law, Wien's displacement law, and infrared and Raman spectroscopy. The energy delivered to the blood circulatory system is based on the photons energy conversion to vibrational modes at the same frequency and phase of molecules, bonds and a group excited by coherent waves at the CO2 bandwidth and knows as core temperature. The core temperature is directly proportional to value of emissive power and inversely proportional to wavelength in range 9.2-9.4 μm (Appendix 4) within the blood circulatory system and can determine the blood energetic parameters compared with mean value of control cases. The coherent waves emitted from the carbon dioxide via TRBS have an irreplaceable role in respiratory physiology. Some diseases can be explained as a deficit of coherent energy, for example infant sudden death—is a sudden energy deficit, Autism Spectrum Disorder (ASD)—represents a partial deficit that occurs during developmental onset, Alzheimer disease—is a severe energy deficit seen in aging, Acquired Immune Deficiency Syndrome (AIDS), —is an acquired deficit. Significant deficit of the infrared energy in CO₂ bandwidth is cause of death of mammalian organism regardless of mechanism leading to deficit. All these disorders have the potential to be treated with optoelectronics. [Appendix 2]

### APPENDIX AMENDMENTS

### Appendix 1

### Power Output Evaluation for TRBS Unit

The lungs are located inside the thorax, covered by pleura with trachea that serves like a small hole entrance to a large cavity, that is maintained at equilibrium, and complies with black body requirements. The power output evaluation of TRBS is based on a heat radiation (temperature of black body) equal to (T) ˜38° C. from the lung surface, calculated from the Stefan-Boltzmann law. The lungs are quite similar to a truncated cone. Using the approximately an adult lungs dimensions the following equation for truncated cone surface (A) is applied:

A=πth (r₁+r₂)

h=25 cm, r₁=10 cm, r₂=5 cm

A=0.11 m²

Approximately surface value of two lungs (A₂):

A₂=0.22 m²

Stefan-Boltzmann law equation for (T)=38° C.:

∈(T)=A₂ σ T⁴

σ=5.670·10⁻⁸ W/(m²·K⁴)

T=273 K+38° C.=311 K

A₂=0.22 m²

Emissive power value for A₂:

∈(T)=117 W/m²

Total number of TRBS units=9.6×10⁸

Approximately emissive power for one TRBS unit:

1 TRBS unit=0.12 μW/m²

### Appendix 2

### Method of Adjusting the Core Temperature Deficit

Body temperature is usually maintained near a constant level of 36.5-37.5° C. (98-100° F.) through biologic homeostasis or thermoregulation. As body temperature decreases, characteristic symptoms occur such as mental confusion and shivering. Even at mild hypothermia, mental confusion appears. The thermal homeostasis of mammals is the key factor supporting correct brain function, but the long term subtle lower core temperature can be a risk factor for brain development and functioning. External factors in the environment, diet, or lifestyle can be a significant source of body temperature variability, which is characteristic for autism spectrum disorders (ASD) problems, and constituted the basis of some treatments. [35] [36] The rapid behavioral changes that have been reported in some children with ASDs when they have a fever, suggest that dysfunctional neural networks in ASDs might be intact but nascent, and that knowledge of the reasons for improvement during fever might elucidate the neurobiological basis of ASDs. In the past few decades, parents and clinicians have reported that behaviors of children with ASDs tend to improve, sometimes dramatically, during febrile episodes. The degrees of fever required to evoke behavior changes in children with ASDs have varied among children and types of infection. Improvements have appeared before or soon after the onset of fever and have subsided within 1-3 days after the fever was gone. It has been suggested that maximal improvements in autistic behaviors may occur at elevations of 1.5-2.5° C. (2.7-4.5° F.) in body temperature. [37] The similar problem of core temperature deficits has been described in a mouse model, where a decrease in temperature during anesthesia leads to overt tau hyperphosphorylation in the brain similar to Alzheimer disease tauopathy. [32][33][34] Even the aging process is characterized by decrease of core temperature [35], tauopathy and related symptoms, e.g., malfunction of ˜34 genes which are responsible for skin collagen synthesis. The mean value of core temperature measured over the long term (i.e., months) can help to evaluate the order of magnitude of a subtle deficit.

The aims of the laser therapy are reverse the energy deficit via full restoration of the core temperature pattern bases on circadian rhythm and mean emissive power of human lungs (TRBS) age related or younger healthy persons. The calculation of core temperature deficit is based on Stefan-Boltzmann Law (Appendix 1) and Wien's Displacement Law (Appendix 4) and 24 h measurements of core temperature especially between 4 pm and 6 pm regards to the highest temperature of circadian rhythm. Duration of treatment should be based on the mean duration of regular pregnancy which is the energetic marker of fetus maturity depends on regular maternal core temperature.

The details of adjusting the core temperature deficit:
The blood contains a lattice of harmonic oscillators which are subtly coupled and can be present as modes of vibration e.g., CO₂ (spectral range 4.25-14.99 μm of wavelength), NaCl (spectral range 0.25-16 μm of wavelength), KCl (spectral range 0.21-20 μm of wavelength), and glucose (spectral range 8.5-10 μm of wavelength), which will support infrared resonant vibrational excitation atoms and molecules and bonds in CO₂ bandwidth and increase the effectiveness of carrying the energy to the whole body (infrared waves as electromagnetic waves hence characterize by transverse modes).
Glucose and CO₂, which are especially supported by the presence of NaCl and KCl, can penetrate through the blood-brain barrier, carrying vibrational energy (electromagnetic waves at 9.2-9.4 μm bandwidth) deeper to the brain's neurons powering the genes as well.
The laser beam must be decollimated on the end of each Polycrystalline Infra-Red (PIR-) fibers (e.g., rays dispersal by ZnSe lens) prevents a damage of any surrounded cells.
The characteristic of PIR-Fiber Cables is:
   High transmittance from 4 μm up to 18 μm
   High flexibility and no toxicity
   Suitable for CO₂-laser power delivery up to 50 W
   Low attenuation at 10.6 μm (0.1-0.5 dB/m)
   Standard fiber diameters from 0.3 to 1.0 mm
   No aging effect
The exposure area will warm up to the required temperature but less than or equal to 40° C. thereby protecting blood proteins and brain function.
The temperature elevations will obtain by using continuous waves of carbon dioxide bandwidth at 9.2-9.4 μm to match the proper core temperature. (Appendix 4) The low-power tunable carbon dioxide laser with tunable attenuator and thirty three PIR-fibers (3×11) should be connected perpendicular to the long axis of tube with effective transmission window for 9.2-9.4 μm wavelength. The tube with PIR-fibers module should be connected to venues system e.g., similar to connection of dialysis machine or any cardiopulmonary bypass (CPB) for adjusting the core temperature value.
The special pattern of fixation to PIR-fibers on the tube's wall should be applied for better exposure of blood flows:
   every three PIR-fibers should be connected to the tube in the same perpendicular plane (to the long axis of tube) at central convex angles equals 3×120°=360 °,
   the equal distance between every set of three PIR-fibers)(3×120° which lies in the eleven different parallel planes should be convenient for set-up
   for uniform distribution of the infrared waves to the blood, every set of three PIR-fibers should be turn at clockwise of 10° (one by one, each plane related to previous plane, to all eleven) to obtain a helix-like pattern.

### Appendix 3

Tissue metabolism=↑CO₂→venous transport of CO₂ to alveolar space→TRBS=↑infrared energy emission+Hb stimulation by infrared energy=↑O₂→arterial transport=O₂+infrared energy→tissue metabolism=↑CO₂

APPENDIX 4
TEMPERATURE VALUES CONVERTED TO WAVELENGTHS
(Based on Wien's Displacement Law)

| Temp. in | Wavelength in |
|---|---|
| ° C. | μm |
| 36.0 | 9.3727 |
| 36.1 | 9.3697 |
| 36.2 | 9.3667 |
| 36.3 | 9.3637 |
| 36.4 | 9.3605 |
| 36.5 | 9.3576 |
| 36.6 | 9.3546 |
| 36.7 | 9.3516 |
| 36.8 | 9.3486 |
| 36.9 | 9.3455 |
| 37.0 | 9.3425 |
| 37.1 | 9.3395 |
| 37.2 | 9.3365 |
| 37.3 | 9.3335 |
| 37.4 | 9.3305 |
| 37.5 | 9.3275 |
| 37.6 | 9.3245 |
| 37.7 | 9.3215 |
| 37.8 | 9.3185 |
| 37.9 | 9.3155 |
| 38.0 | 9.3125 |
| 38.1 | 9.3095 |
| 38.2 | 9.3065 |
| 38.3 | 9.3035 |
| 38.4 | 9.3005 |
| 38.5 | 9.2976 |
| 38.6 | 9.2946 |
| 38.7 | 9.2916 |
| 38.8 | 9.2886 |
| 38.9 | 9.2856 |
| 39.0 | 9.2827 |
| 39.1 | 9.2797 |
| 39.2 | 9.2767 |
| 39.3 | 9.2738 |
| 39.4 | 9.2708 |
| 39.5 | 9.2678 |
| 39.6 | 9.2649 |
| 39.7 | 9.2619 |
| 39.8 | 9.2589 |
| 39.9 | 9.2560 |
| 40.0 | 9.2531 |

What I claim as my invention is:

1. The Terminal and Respiratory Bronchioles System (TRBS) is composed of three modules: Tunable microcavity module—in the terminal bronchioles, Compressor module—in the alveolar ducts and respiratory bronchioles, Reservoir module—in the alveolar sacs.

2. (canceled)

3. The thresholdless emission in the terminal bronchioles is based on continuous collisions of cooler CO₂ and warmer N₂ molecules in the metastable vibrational level, within the tunable microcavity that leads to the carbon dioxide population inversion via vibrational excitation necessary for lasing operation in microcavity.

4. The dissociation of water catalyzed by hemoglobin as organic semiconductor excited by photo-vibrational energy transfer within erythrocytes that occurs via TRBS photons energy converts to the vibrational modes and leads to hemoglobin saturation by endogenous oxygen.

5.-7. (canceled)

8. Based on the principles of Debye model of solid body, Planck's Law, Wien's Displacement Law, infrared and Raman spectroscopy, the function of TRBS can be replaced or improved, by use of the low power CO₂ tunable laser with decollimated flux of photons (e.g., ZnSe dispersal lens) that is a substitute for the life sustaining source of energy in range 9.2-9.4 μm of wavelength.
